(19) United States
(12) Patent Application Publication
(10) Pub. No.: US 2025/0325295 A1
(43) Pub. Date: Oct. 23, 2025

(54) SYSTEMS AND DEVICES FOR PERFORMING INCISIONS ON TISSUE

(71) Applicant: EDWARDS LIFESCIENCES CORPORATION, Irvine, CA (US)

(72) Inventors: Yaeer E. Lev, Torrance, CA (US); Farzad Azimpour, Irvine, CA (US); Atiya Makhdoom Ahmad, Irvine, CA (US); Leonardo Paim Nicolau Da Costa, Tampa, FL (US); Amir Yousef Sajjadi, Aliso Viejo, CA (US); Daniel M. Harps, Trabuco Canyon, CA (US); Allen Jeong Keel, Irvine, CA (US)

(73) Assignee: EDWARDS LIFESCIENCES CORPORATION, Irvine, CA (US)

(21) Appl. No.: 19/254,257

(22) Filed: Jun. 30, 2025

Related U.S. Application Data
(63) Continuation of application No. PCT/US2024/013194, filed on Jan. 26, 2024.
(60) Provisional application No. 63/481,974, filed on Jan. 27, 2023.

(51) Int. Cl. A61B 17/3209; A61B 17/00; A61B 17/32; A61B 18/00; A61B 18/08; A61B 18/14; A61M 25/00

(52) U.S. Cl. CPC A61B 17/3209; A61B 18/082; A61B 18/1492; A61M 25/0023; A61B 2017/00557; A61B 2017/00566; A61B 2017/00867; A61B 2017/320044; A61B 2018/00077; A61B 2018/00095; A61B 2018/0022; A61B 2018/00363; A61B 2018/00601; A61B 2018/144

(57) ABSTRACT

Systems and devices for incising tissue are provided. A system can comprise a catheter having a proximal-distal axis extending from a distal end of the catheter. The catheter includes an indentation adjacent to the distal end of the catheter, a vacuum system having one or more vacuum apertures situated within the indentation, where the vacuum apertures are connectable to a vacuum source for providing a vacuum to pull tissue into the indentation, and means for incising tissue connected adjacent to the distal end of the catheter and slidable relative to the indentation, whereby tissue located in the indentation can be incised by sliding the means for incising relative to the indentation while the vacuum system is activated.

### RELATED APPLICATIONS

This application is a continuation application of International Patent Application No. PCT/US2024/013194, filed Jan. 26, 2024, which claims the benefit of U.S. Patent Application No. 63/481,974, filed Jan. 27, 2023, the entireties of each of which are hereby incorporated by reference.

### TECHNOLOGICAL FIELD

The disclosure is generally directed to systems and devices for providing a means to incise tissue, and in some instances to incise an outer tissue layer.

### BACKGROUND

The pericardium, also referred to as the pericardial sac, is double-layered tissue that surrounds the heart and provides it protection. The outer layer of the pericardium is the fibrous pericardium formed of strong connective tissue and the inner layer is the serious pericardium formed of a serous membrane. Between the pericardium and the heart is the pericardial space, which a lubricous space that allows the heart to function without friction.

Heart failure is a condition in which filling and/or ejection of blood from the heart is compromised, leading to ineffective perfusion of the body and accompanying fatigue, dyspnea (difficulty breathing), orthopnea (difficulty breathing while lying flat), arrhythmias (electrical abnormalities), and death. While there are various causes of heart failure, the elastic pericardium can contribute to the low ejection fraction by exerting a compressive force on the heart myocardium. Thus, one way to increase to improve heart function is to relieve the compressive force provided by the pericardium.

### SUMMARY

In one implementation, a system for incising tissue comprises a catheter having a proximal-distal axis, the catheter comprising an indentation, a vacuum system, and a means for incising tissue. The indentation is elongated along the proximal-distal axis. The vacuum system comprises a vacuum supply in connection to a vacuum lumen and set of one or more vacuum apertures, the vacuum lumen extending along the proximal-distal axis and in connection with the set of vacuum apertures, and the vacuum apertures are situated within the indentation.

In some aspects, the means for incising tissue is a blade in connection with the catheter, the blade having a sharp edge and capable of moving in a direction along the proximal-distal axis over the indentation.

In some aspects, the blade comprises a leading-edge point on the sharp edge, wherein the sharp edge curves or angles back from the leading-edge point.

In some aspects, the blade has body that is semi-tubular or shaped like triangular prism with an open face that faces towards the indentation of the catheter when positioned over the indentation.

In some aspects, the means for incising tissue is a wire in connection with the catheter, the wire capable of moving in a direction along the proximal-distal axis over the indentation.

In some aspects, the wire is conductive of heat or electricity.

In some aspects, the catheter further comprises a means for providing a counterforce to the means for providing an incision.

In some aspects, the means for providing a counter force is one of: a lancet extending from the catheter, a prong extending from the catheter, a helix extending from the catheter, a barb attached to the catheter, a clasp attached to the catheter, a jagged edge on the catheter, or a textured surface on the catheter.

In some aspects, the means for incising tissue is a first blade in connection with the catheter, the first blade having a sharp edge and capable of rotating in a direction perpendicular the proximal-distal axis over the indentation.

In some aspects, the catheter further comprises a rotating member with a blunt edge in connection with the catheter for compressing tissue.

In some aspects, the system further comprises a second blade in connection with the catheter, the second blade having a sharp edge and capable of rotating in a direction perpendicular the proximal-distal axis and opposite to the first blade.

In some aspects, the size of the indentation predetermines an amount of tissue to be excised.

In some aspects, the catheter further comprises an expandable balloon.

In some aspects, the catheter further comprises a rotatable disc within the indentations. A subset of the vacuum apertures of the set of vacuum apertures are situated within the rotatable disc.

In some aspects, the vacuum system further comprises a slidable member that can slide along the proximal-distal axis for occluding one or more vacuum apertures of the set of vacuum apertures.

In some aspects, the system further comprises a control system. The catheter and the control system are a part of a transcatheter system such that the control system is in operable connection with the catheter and is capable of distally advancing and proximally retracting the catheter along the proximal-distal axis. The control system is further capable of operating the means for incising tissue.

In one implementation, a catheter is delivered to an inner space between a first tissue layer and a second tissue layer. The catheter has a proximal-distal axis and comprises an indentation, a vacuum system, and a means for incising tissue. The indentation is elongated along the proximal-distal axis. The vacuum system comprises a vacuum supply in connection to a vacuum lumen and set of one or more vacuum apertures, the vacuum lumen extending along the proximal-distal axis and in connection with the set of vacuum apertures, and the vacuum apertures are situated within the indentation. The vacuum system is activated such that a portion the first tissue layer is grasped within the indentation of the catheter. The first tissue is incised via the means for incising tissue.

In some aspects, the means for incising tissue is a blade in connection with the catheter. Incising the first tissue comprises moving the blade over the indentation along the proximal-distal axis.

In some aspects, the means for incising tissue is a wire in connection with the catheter. Incising the first tissue comprises moving the wire over indentation along the proximal-distal axis.

In some aspects, the means for incising tissue is a blade in connection with the catheter. Incising the first tissue comprises rotating the blade over indentation in a direction perpendicular to the proximal-distal axis.

In some aspects, the catheter is removed from the inner space. The vacuum system remains activated, and the incised tissue remains within the indentation of the catheter.

In some aspects, the first tissue is a pericardium, and the second tissue is a myocardium.

In some aspects, the inner space between the pericardium and the myocardium is reached via a subxiphoid approach.

In one implementation, a system for incising tissue comprises a catheter comprising a lumen, having a proximal-distal axis, and the lumen is composed of a shape-memory material. The shape-memory lumen of the catheter comprises a looped curve at the distal end of the catheter. The system further comprises a tool comprising a blade or wire at the distal end of the tool. The tool is positioned within the lumen.

In some aspects, the catheter comprises an aperture on a side of the catheter that is located within the looped curve, the aperture facing towards the center of the loop. The tool optionally comprises a flexible hinge proximal to the blade or wire, or the blade or wire is comprised of shape-memory material. The flexible hinge or shape-memory material allows the blade or wire to align with the aperture within the looped curve.

In some aspects, when the blade or wire is aligned with the aperture within the looped curve, the flexible hinge is flexed at or near 90-degrees.

In some aspects, the system further comprises a vacuum system that provides a vacuum via the lumen of the catheter.

In some aspects, the system further comprises an outer sheath that confines the catheter. In some aspects, the outer sheath confines the shape-memory lumen to the proximal-distal axis of the outer sheath.

In some aspects, the system further comprises a control system. The catheter and the control system are a part of a transcatheter system such that the control system is in operable connection with the catheter and the tool and is capable of independently distally advancing and proximally retracting the catheter and the tool along the proximal-distal axis.

In one implementation, a catheter and a tool are delivered to an inner space between a first tissue layer and a second tissue layer. The catheter comprises a lumen, has a proximal-distal axis, and is composed of a shape-memory material, the shape-memory of the catheter comprises a looped curve at the distal end of the catheter. The catheter is confined within an outer sheath. The tool is within the lumen of the catheter, the tool comprising a blade at the distal end and a flexible hinge proximal to the blade. The catheter is initially distally advanced out of outer sheath a distance such that the catheter bends based on in its shape-memory such that an aperture at the distal end of catheter faces toward an inner surface of the first tissue layer. The tool is distally advanced such that the blade advances out of the aperture at the distal end of the catheter to penetrate and traverse the first the first tissue to yield a first incision. The catheter if further distally advanced out of the outer sheath a distance such that the looped curve is formed. The advancing of the catheter results in an aperture within a side wall of the catheter is aligned with the plane of the first tissue layer, the aperture within the side wall is located within and faces the center of the looped curve portion of the catheter. A sharp edge of the blade is aligned such that it points towards the aperture within the side wall of the catheter. The outer sheath, the catheter, and the tool are concurrently proximally retracted such that they maintain relative spacing along the proximal-distal axis to incise the tissue.

In some aspects, a vacuum is applied through the lumen of the catheter to grasp the inner surface of the first tissue layer prior to distally advancing the tool.

In some aspects, a vacuum is applied through the lumen of the catheter to grasp an outer surface of the first tissue layer prior to proximally retracting the outer sheath, the catheter, and the tool concurrently.

In some aspects, the first tissue is a pericardium, and the second tissue is a myocardium.

In some aspects, the inner space between the pericardium and the myocardium is reached via a subxiphoid approach.

### BRIEF DESCRIPTION OF THE DRAWINGS

The description and claims will be more fully understood with reference to the following figures and data graphs, which are presented as examples of the disclosure and should not be construed as a complete recitation of the scope of the disclosure.

FIGS. 1A and 1B provide views of an exemplary system for incising tissue utilizing a catheter and vacuum system. FIG. 1A is peripheral view. FIG. 1B is an exploded view.

FIGS. 2A and 2B provide detailed views of the exemplary system in FIGS. 1A and 1B. FIG. 2A is peripheral view. FIG. 2B is a cross-sectional view.

FIGS. 2C, 2D, and 2E provide detailed views of different implementations of the exemplary system that incorporate different components for providing a counterforce.

FIGS. 3A, 3B, and 3C provide various examples of blades for use in a system for incising tissue. FIGS. 3A and 3B provide an exemplary blade having a semi-tubular shape and rounded leading edge. FIG. 3A is peripheral view. FIG. 3B is a front view. FIG. 3C provides a peripheral view of an exemplary blade having a flat shape.

FIGS. 3D and 3E provide detailed views of the blades within FIGS. 3A and 3C installed within in a system for incising tissue.

FIGS. 4A, 4B, and 4C provide views of an exemplary system for incising tissue utilizing a wire as a means for incising tissue. FIG. 4A provides a top view. FIG. 4B provides a peripheral view. FIG. 4C provides a peripheral view in which the wire is partially retracted.

FIGS. 5A, 5B, and 5C provide views of an exemplary system for incising tissue utilizing rotational blade. FIG. 5A provides a peripheral view. FIG. 5B provides a cross-sectional view. FIG. 5C provides a cross sectional view in which the blade has been rotated.

FIGS. 6A, 6B, 6C, and 6D provide cross-sectional views of an exemplary system for incising tissue utilizing rotational blade and blunt-edged compressor.

FIGS. 7A and 7B provide detailed peripheral views of an exemplary system for incising tissue that incorporates a set of two balloons.

FIGS. 7C, 7D, and 7E provide top vies of an exemplary system for incising tissue that incorporates a rotational disc.

FIGS. 8A, 8B, and 8C provide views of an exemplary system for incising tissue utilizing a memory-shape catheter and tool with a blade. FIG. 8A provides a side view in which an outer sheath keeps the memory-shape catheter linear. FIG. 8B provides a side view in which the memory-shape catheter is advanced out of an outer sheath allow a looped curl shape. FIG. 8C provides an exploded view.

FIGS. 9A to 9F provide views of an exemplary method of using an exemplary system for incising tissue utilizing a catheter, a vacuum system, and a blade.

FIGS. 10A to 10D provide views of an exemplary method of using an exemplary system for incising tissue utilizing a memory-shape catheter and tool with a blade.

### DETAILED DESCRIPTION

The current disclosure details systems and devices for incising tissue. The systems and devices can utilize a catheter design for minimal invasiveness within the body. Accordingly, systems and devices can comprise a catheter that provides a means for translocating tools for incising tissue. In many implementations, the systems and devices can be utilized within an inner space between two tissues and provides a means for incising one of the two tissue layers, and especially the outer tissue layer. Further, the incision of tissue can provide an access to the inner space or release pressure caused by a tissue layer.

Systems and devices are directed to catheter systems that can comprise a catheter having an enlarged aperture that spans along the distal-proximal axis, the enlarged aperture being near the distal end of the catheter system. Within the elongated aperture are a set of one or more apertures for applying a vacuum. The set of vacuum catheters can be utilized for grasping a tissue layer via a vacuum force. The catheter systems can further comprise a means for incising tissue. The means for cutting tissue can be a sharp-edged blade, a wire, or other tool capable of incising through tissue. In some implementations, the incising action is moving the cutting means in a direction along the distal-proximal axis, or in other words along the length of the elongated aperture. In some implementations, the incising action is moving the cutting means in a direction that is perpendicular to the distal-proximal axis, or in other words across the width of the elongated aperture.

Systems and devices are directed to catheter systems that can comprise a tubular catheter within an outer sheath, and the catheter having an incising means therein. The incising means can be a sharp-edged blade with piercing ability (e.g., like a scalpel). The catheter can have shape-memory such that when it is advanced it can bend upward and then curl in on itself. An aperture can be included on a side of the tubular catheter within the curled portion. An incising means can be provided to pierce the tissue to allow the catheter to traverse therethrough. An incising means can also be provided to lineup with the side aperture to incise tissue as the catheter is pulled in a direction along the tissue. In some implementations, a vacuum is provided through the lumen of the tubular catheter, which can be utilized to grasp and stabilize tissue for incising therethrough.

The described systems, devices, and methods should not be construed as limiting in any way. Instead, the present disclosure is directed toward all novel and nonobvious features and aspects of the various disclosed systems and devices, alone and in various combinations and sub-combinations with one another. The disclosed systems, devices, and methods are not limited to any specific aspect, feature, or combination thereof, nor do the disclosed systems, devices, and methods require that any one or more specific advantages be present or problems be solved.

Various examples of tissue incising systems and components thereof are disclosed herein, and any combination of these examples can be made unless specifically excluded. For example, the various means for incising can be used with any catheter system, even if a specific combination is not explicitly described. Likewise, the different constructions and features of tissue incising systems can be mixed and matched, such as by combining any tool for incising tissue, any tool for grasping tissue, and any system for providing traversing to the site of incision, any device, even if not explicitly disclosed. In short, individual components of the disclosed systems can be combined unless mutually exclusive or physically impossible.

Although the operations of some of the disclosed methods are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods, systems, and apparatus can be used in conjunction with other systems, methods, and apparatus.

The terms “proximal” and “distal” as used throughout the description relate to a catheter system axis, in which the end where the procedure is performed is the distal end and the opposite end where the catheter system is controlled is the proximal end. Accordingly, the distal end of the catheter system is the leading end that first traverses into the body and first reaches the procedure site. Conversely, the proximal end of the catheter system is the portion that remains extracorporeal. Likewise, a distal movement along the catheter axis would be movement of a component in a direction towards a site of procedure and a proximal movement along the catheter axis would be movement of a component in an opposite direction. Although these terms have a relationship with a site of procedure, it is to be understood that these terms are used for reference and the site of procedure does not need to be present when interpreting the components or movements of the devices and systems described herein.

Various systems and devices disclosed herein for incising tissue are utilized for the purpose of performing a procedure within a recipient. Recipients include (but are not limited to) patients, animal models, cadavers, or anthropomorphic phantoms. Accordingly, in addition to methods of treating patients, the systems and devices can be utilized in training or other practice procedures upon animal models, cadavers, or anthropomorphic phantoms.

The described systems and devices can be sterilized, which can be performed using gamma irradiation, gas plasma, aldehydes, ethylene oxide, and/or e-beam. The systems or devices can be further treated with a formaldehyde bioburden reduction process. After preparation, the systems and devices can be stored within a container, which can be hermetically sealed or otherwise kept sterile.

### Systems and Devices for Incising Tissue

Systems and devices for incising tissue can comprise a catheter system such that it can be utilized within minimally invasive procedures. The systems and devices can be utilized to traverse into an inner space between two tissues to incise one of the two tissues. The systems and devices can comprise a vacuum supply to provide the vacuum force via a set of vacuum apertures to grasp a tissue to be incised. The systems and devices can further comprise a means for incising tissue, such as (for example), a sharp edge, a wire, or any other tool for cutting tissue. In some implementations, the means for incising tissue is conductive such that it can provide electricity and/or heat to enhance the incision. The systems and devices can be manufactured to specifications to extract a specific amount of tissue, as determined by the length of tissue captured by the vacuum and the width of the means for incising tissue.

The systems and devices for incising tissue can be utilized on any tissue, or any two tissues in which an inner space can be accessed. In many instances, the systems and devices are utilized to access an inner space between connective tissue and muscular tissue. One such example an inner space to be accessed is the pericardial space between the pericardium and the myocardium. The pericardium is a connective tissue that surrounds the muscular heart tissue. For example, in some instances, the systems and devices are utilized to traverse into the pericardial space between the pericardium and the myocardium and utilized to incise a portion of the pericardium. Any catheter approach can be utilized to reach the two tissues and inner space. For example, a subxiphoid approach can be utilized to access the pericardial space.

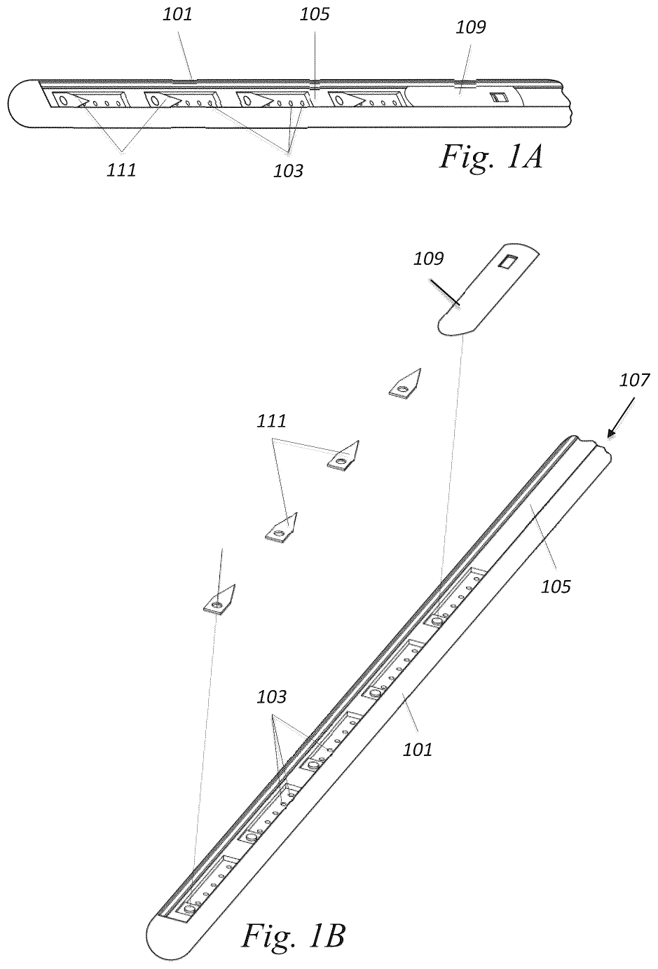

Provided in FIGS. 1A and 1B is an example of a system for incising tissue, the system is utilized with a transcatheter system and can be utilized within an inner space between two tissues. The system comprises a catheter 101, which includes a vacuum supply system utilized to grasp tissue. As shown, the vacuum supply system comprises a set of apertures 103 that are provided along the catheter. In some implementations, and as shown, the vacuum apertures 103 are provided linearly along the proximal-distal axis. The vacuum apertures, however, can be provided in pattern provided that they have the capability of grasping tissue via the vacuum force supplied through the apertures. The set of vacuum apertures reside within an indentation 105 that is elongated along the proximal-distal axis of catheter 101. The length and width of indentation 105 can correspond to the length of tissue to be extracted, based on the amount of tissue grasped therein. Accordingly, the system for incising tissue, catheter 101 and indentation 105 can be sized for incision and removal of a predetermined amount of tissue.

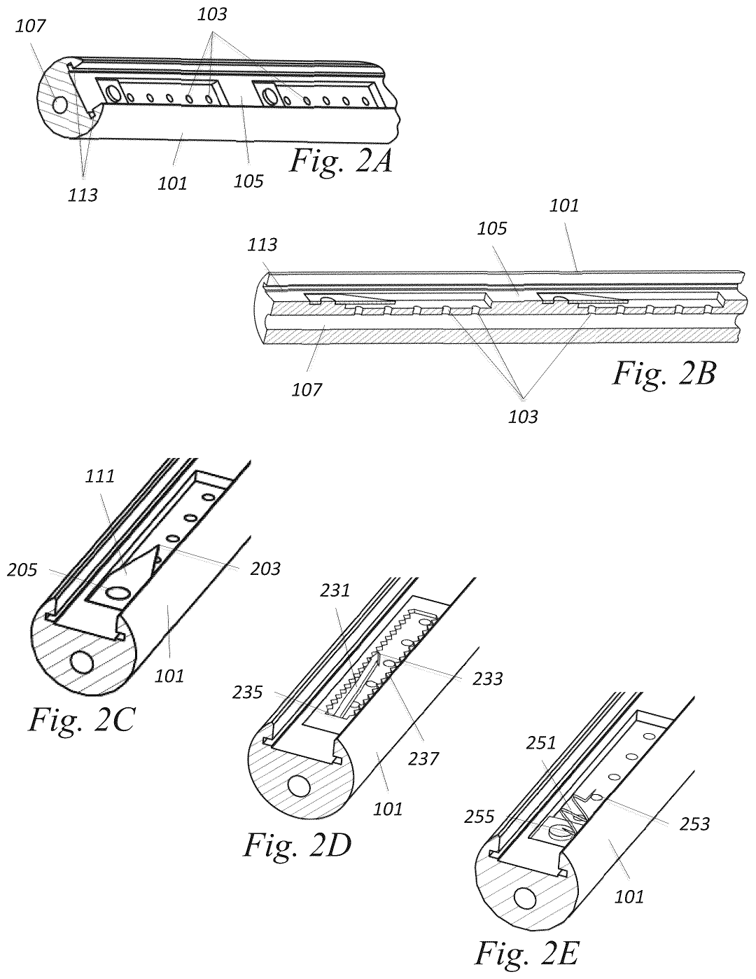

Within the catheter is an inner lumen 107 (see cross-section in FIG. 2A) for connecting the vacuum apertures with a vacuum supply provided at the proximal end of the transcatheter system (not shown). In some implementations, the system further includes an occluding mechanism to occlude one or more vacuum apertures. For example, the occluding mechanism can be slidable member within the inner lumen that can be advanced in the distal direction and retreated in the proximal direction. The slidable member can slide across the apertures at the point of connection with inner lumen such that the connection is occluded.

The system for incising tissue comprises a means for incising tissue. As shown in the example within FIGS. 1A and 1B, the system comprises a blade 109 that is able to be slid in the distal direction. Blade 109 includes a sharp edge on its distal end such that when the blade slides in the distal direction, the blade can incise tissue that is grasped by the supplied vacuum. Although blade 109 is shown to incise tissue by sliding in the distal direction, various implementations can utilize a means for incising that moves in the proximal direction or in a direction perpendicular to the proximal-distal axis. To assist the incision of tissue, in some implementations, the system can comprise a means for providing a counterforce to the incising action. For example, as shown, the system can comprise a set of one or more lancets 111 within the indentation 105 capable of penetrating and grasping tissue therein. The lancets can include a base in connection with catheter 101 and a tip that extends in the direction opposite to the movement of the incising action. Accordingly, when blade 109 slides in the distal direction, lancets 111 having a tip extending towards the proximal direction can grasp tissue and provide a counter force to the movement of the blade.

FIGS. 2A and 2B provide a more detailed view of catheter 101 in two-sectional views. Accordingly, a vacuum supply is provided at the proximal end of the system (not shown) that is connection with inner lumen 107 to provide a vacuum force to indentation 105 via vacuum apertures 103. Along each side of indentation 105 is a groove 113 in which a means for incising can fit within such that it can slide along the proximal-distal axis.

FIGS. 2C, 2D, and 2E provide examples of various components that can be utilized as a counterforce to be utilized with a means for incising that utilizes a push or pull mechanism in along the proximal-distal axis (e.g., slidable blade, wire). FIG. 2C provides an example of a lancet 111, which has a pointed tip 203 that is pointed in the opposite direction of the means for incising tissue. At an end opposite of pointed tip 203 is attachment point 205 of the lancet to attach to catheter 101. Edges 207 of lancet 111 are angled back towards attachment point 205, which can help with grasping and securing tissue. FIG. 2D provides a prong 231 that is extended with a pointed tip 233, and further includes an attachment point 235 at an end opposite of the pointed tip. Prong 231 is extended and tip 233 is pointed in the opposite direction of the means for incising tissue. Also depicted in FIG. 2D are jagged edges 237 that are distributed along the proximal-distal axis, which can be utilized to help grasp and hold tissue to provide a counterforce. Although jagged edges 237 are depicted with prong 231, it should be understood that each of these means of providing a counterforce can be used independently or with any other means for providing a counterforce. FIG. 2E provides a helix 251 with a pointed tip 253, and further includes an attachment point 255 at an end opposite of the pointed tip. Helix 251 is extended and tip 233 is pointed in the opposite direction of the means for incising tissue. Helix 251 can be rigid or can flex to provide a spring force. It should be understood that any means for providing a counterforce can be utilized, even if not depicted in an illustrated example. Other counterforces can include (but are not limited to) barbs, spikes, clasps, and textured patterns on the catheter. It should also be understood that one or more means for providing a counter can be combined or utilized within the same catheter.

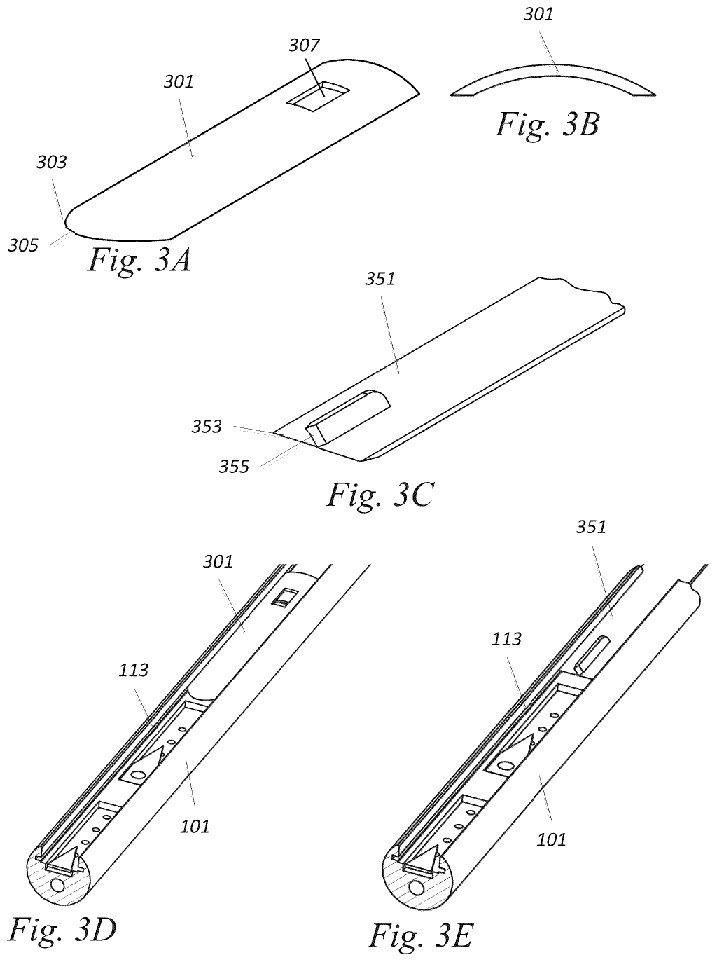

FIGS. 3A, 3B, and 3C provide examples of various blades that can be utilized in a system for incising tissue. FIGS. 3A and 3B are two views of a blade 301 with sharp edge 303 with a leading-edge point 305, where edge 303 curves back from point 305 to yield a curved sharp edge. Blade 301 also has a semi-tubular shape with a rounded face at the top and an open flat face at the bottom. The curve of edge 303 and the semi-tubular shape allows the blade to penetrate through a tissue at leading-edge point 305 and then push the tissue downward as the blade traverses through the tissue. Various alternative blade designs can be implemented and still yield the same benefit of penetrating the tissue then pushing it downward. For example, in some implementations, the sharp edge can have a leading-point were the edge angled back from the point. Likewise, in some implementations, instead of a semi-tubular body, the blade can have triangular prism design with an open face at the bottom.

Provided in FIG. 3C is another alternative design in which blade 351 is flat with a sharp edge 353 that is straight. Blade 351 further includes a leading-edge 355 that extends upward and forward from the top surface of the sharp edge of the flat blade. Leading-edge 355 has two angled faces extending down and back away from the edge. Because this leading-edge design extends upward and forward, can penetrate tissue and then push the tissue downward as it traverses through the tissue.

Regardless of the design of the blade, the blade can be composed of any material useful in for surgical procedures, such as (for example) stainless steel or high carbon steel.

FIGS. 3D and 3E provide examples of blades installed within the incising system. In FIG. 3D, the side edges of blade 301 fit within grooves 113 of catheter 101 such that the blade can move in a direction along the proximal-distal axis. A pusher can be utilized for moving blade 301 in the proximal and distal directions. The pusher can be a rod, strut, or any other component for pushing a blade. Blade 301 can include a means for operatively connecting with a pusher. As shown, blade includes a notch 307 in which pusher engages to form an operable connection. Pusher can extend proximally to the transcatheter controller or be in operable connection with the transcatheter controller such that an operator can control the movement of blade 301. In FIG. 3E, the side edges of blade 351 fit within grooves 113 of catheter 101 such that the blade can move in a direction along the proximal-distal axis. Instead of a pusher, the back end of the blade extends proximally to the transcatheter controller or is operably connected with the transcatheter controller such that an operator can control the movement of blade 351. It should be understood that any blade design can be utilized with any means for moving the blade unless such combination is operatively impossible and should not be limited to the exact arrangements as portrayed.

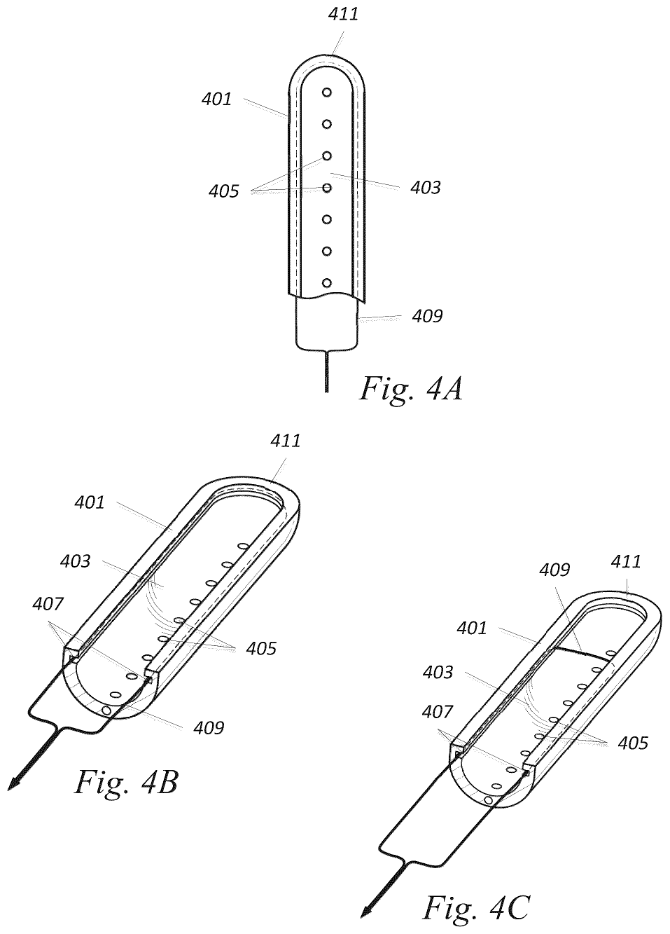

Provided in FIGS. 4A to 4C is an example of a system for incising tissue in which the means for incising tissue is a wire. Catheter 401 comprises an indentation 403 in which a set of vacuum apertures 405 are provided. Along each side wall of indentation 403 is a groove 407 in which a wire 409 can situate therein. Wire 409 can be looped such that the distal end 411 of the wire crosses indentation 403. Further, groove 407 can extend to the distal end of the catheter, allowing the distal end 411 of wire 409 to situate therein when the catheter is being delivered to the site of incision. After a vacuum force grasps a tissue via vacuum apertures 405, wire 409 can be pulled proximally to incise through the tissue (FIG. 4C). Wire 409 can conduct heat and/or electricity to assist in incising the tissue. The wire 409 can be composed of any material capable of cutting through tissue and/or capable of conductance, such as (for example) stainless steel, nitinol, copper alloys, nickel alloys, gold, tungsten, and titanium.

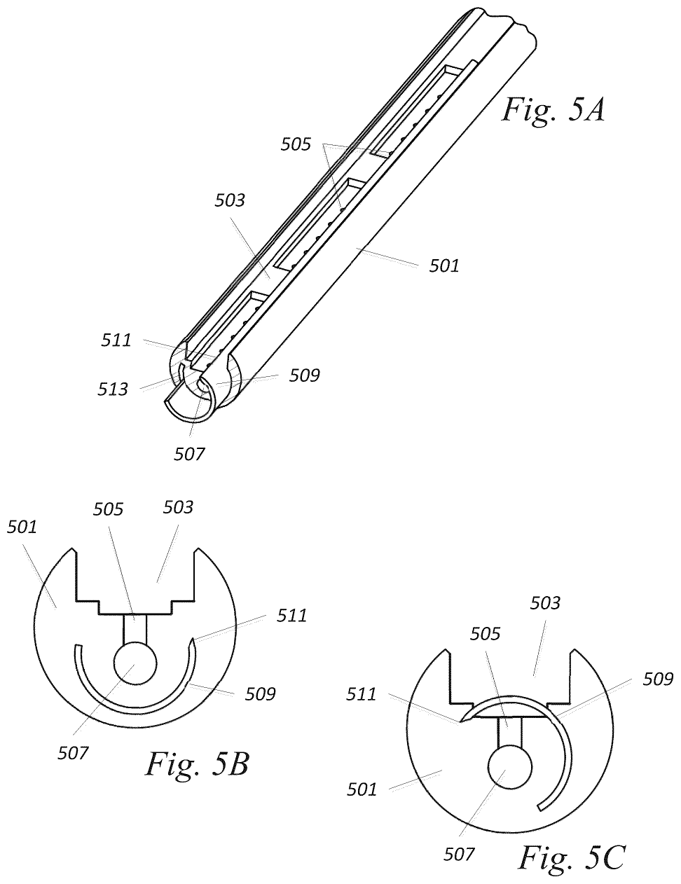

Provided in FIGS. 5A to 5C is an example of a system for incising tissue in which the means for incising tissue moves in a direction perpendicular to the proximal-distal axis via a rotating member. In this example, the system comprises a catheter 501, an indentation 503 within the catheter, and a plurality of vacuum apertures 505 situated within the indentation. The plurality of vacuum apertures 505 are in connection with an inner lumen 507 and a vacuum supply to provide a vacuum force for grasping tissue in a similar manner to the example described in FIGS. 1A and 1B. Catheter 501 further comprises a blade 509 having a semi-tubular shape and a sharp edge 511 that extend along the proximal-distal axis. Blade 509 can be stored within a cavity 513 while the catheter traverses through the body to the incision site. Blade 509 is in operable connection with the transcatheter controller at the proximal end of the system. As shown in FIG. 5C, blade 509 can be turned in a circular direction via the transcatheter controller such that the blade crosses indentation 503, led by sharp edge 511. The turning of blade 509 allows sharp edge 511 to penetrate and traverse a tissue that would be grasped within indentation 503, incising a strip of tissue in the process. Although FIGS. 5A to 5C show blade 509 turning in a counter-clockwise direction, it should be understood that alternative designs could have a blade turning in a clockwise direction as well. The blade can be composed of any material useful in for surgical procedures, such as (for example) stainless steel or high carbon steel.

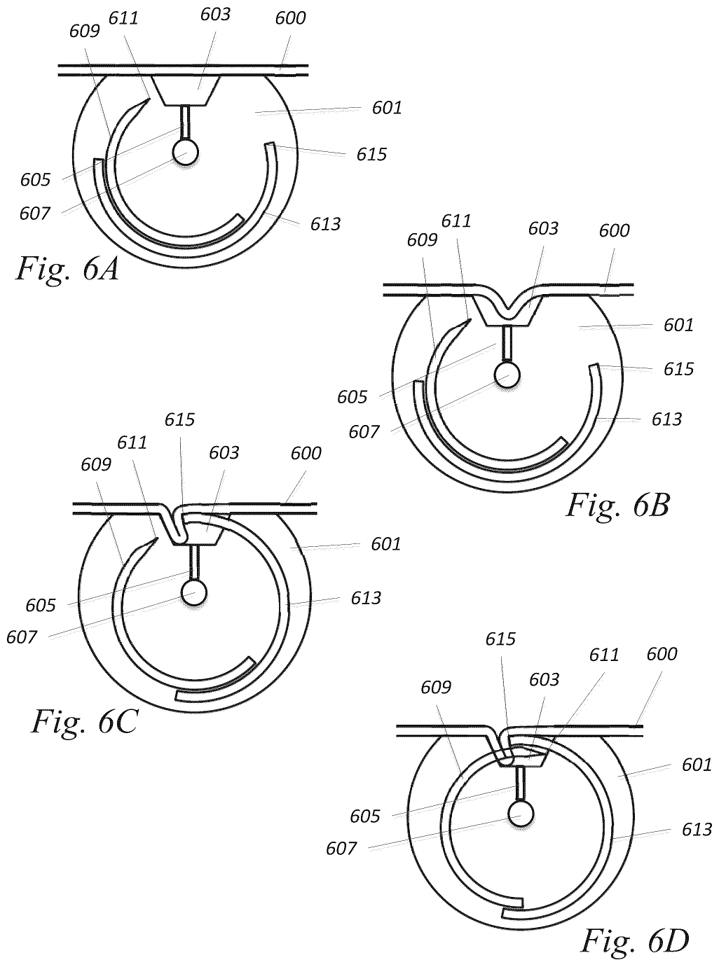

FIGS. 6A to 6D provide an example of a system for incising tissue having two rotating members that move in a direction perpendicular to the proximal-distal axis, where at least one of the rotating members provides a means for incising tissue. In this example, the system comprises a catheter 601, an indentation 603 within the catheter, and a plurality of vacuum apertures 605 situated within the indentation. The plurality of vacuum apertures 605 are in connection with an inner lumen 607 and a vacuum supply to provide a vacuum force for grasping tissue in a similar manner to the example described in FIGS. 1A and 1B. Catheter 601 further comprises a blade 609 having a sharp edge 611 and a compressor 613 having a blunt edge 615. Blade 609 and compressor 613 each have a semi-tubular shape that extends along the proximal-distal axis. Sharp edge 611 and blunt edge 615 also extend along the proximal-distal axis. Blade 609 and compressor 613 can each be stored within a cavity while the catheter traverses through the body to the incision site. Blade 609 and compressor 613 are each in operable connection with the transcatheter controller at the proximal end of the system.

For purposes of explaining the mechanism of the system for incising tissue having two rotating members, FIGS. 6A to 6D depict a tissue 600, however it should be understood that the tissue is not part of the system. Catheter 601 is brought in proximity to tissue 600 with indentation 603 facing the tissue (FIG. 6A). The vacuum supply grasps the tissue via vacuum apertures 605 (FIG. 6B). Compressor 613 rotates in a circular direction to compress tissue 600 against a side wall of indentation 603 (FIG. 6C). Blade 609 rotates in a circular direction to penetrate and traverse through tissue 600, incising a strip of tissue in the process (FIG. 6D). Although FIGS. 6A to 6D show the compressor rotating in one circular direction and the blade rotating in an opposite circular direction, it is to be understood that the compressor and blade can rotate in the same direction as long as the blade can penetrate and traverse through the tissue. Likewise, the compressor and blade are not limited to any particular clockwise or counter-clockwise direction and thus can rotate in either direction.

Various designs of a system for incising tissue having two or more rotating members are contemplated. In some implementations, the system for incising tissue comprises two blades, a first blade rotating in a first direction and second blade rotating in a second direction opposite of the first direction. Accordingly, systems comprising two blades can each rotate towards a grasped tissue within the indentation. Each blade can penetrate and traverse the tissue such that the first blade traverses through the tissue just above the second blade in a scissor-like manner to perform the incision. In some implementations, the system for incising tissue comprises two compressors and a blade, a first compressor rotating in a first direction and second compressor rotating in a second direction opposite of the first direction. The two compressors can rotate in the same plane such that the blunt end of the first compressor aligns and meets the blunt end of the second compressor within the indentation when rotated. Accordingly, the first compressor and the second compressor can pinch a tissue within the indentation. With the tissue pinched, the blade can penetrate and traverse the tissue to perform the incision.

Various designs can utilize various combinations of blades and/or compressors, as long as at least one blade is provided for performing the incision. In some implementations, a system comprises a single blade (see FIGS. 5A to 5C). In some implementations, a system comprises a single blade and a single compressor (see FIGS. 6A to 6D). In some implementations, a system comprises two blades. In some implementations, a system comprises two compressors and a single blade. In some implementations, a system comprises a single compressor and two blades. In some implementations, a system comprises two compressors and two blades.

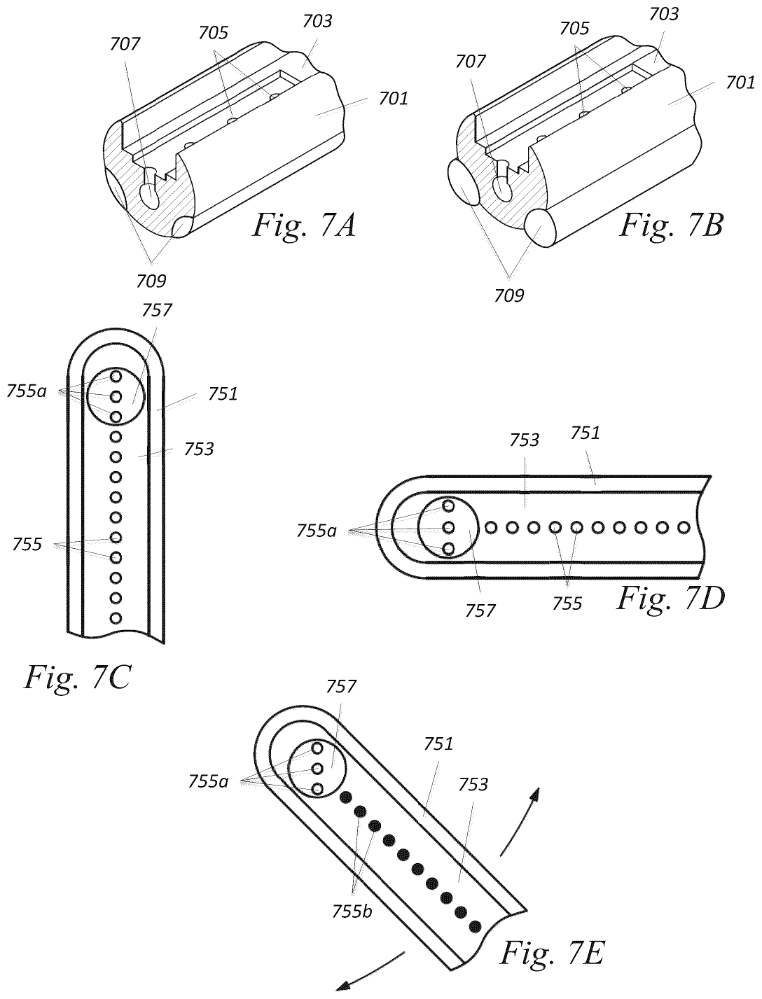

Some implementations of a system for incising tissue can comprise an expandable member, which can be utilized with any system having any means for performing the incision. When the system is between two tissue layers, the expandable member can elevate the system such that the indentation and vacuum apertures are in closer proximity to the tissue to be grasped. Provided in FIGS. 7A and 7B is an example of a system for incising tissue that incorporates a set of balloons that are expandable. In this example, the system comprises a catheter 701, an indentation 703 within the catheter, and a plurality of vacuum apertures 705 situated within the indentation. The plurality of vacuum apertures 705 are in connection with an inner lumen 707 and a vacuum supply to provide a vacuum force for grasping tissue in a similar manner to the example described in FIGS. 1A and 1B. Catheter 701 further comprises a set of two balloons 709 that are expandable. FIG. 7B portrays the set of balloons in the expanded state. An air supply can be utilized to expand the set of balloons 709, which can be delivered via the transcatheter system. Expansion of the set of balloons 709 increases the overall profile of catheter 701, which can be useful for bringing indentation 703 in proximity to the tissue that is to be incised.

It can be desirable to change the axis of the catheter in relationship to the tissue to be incised. Accordingly, various implementations of a system for incising tissue can comprise a means for adjusting the angle of the axis of the catheter. Provided in FIGS. 7C to 7E is an example a system for incising tissue that incorporates a rotatable disc having a subset of vacuum apertures. In this example, the system comprises a catheter 751, an indentation 753 within the catheter, and a plurality of vacuum apertures 755 situated within the indentation. The plurality of vacuum apertures 755 are in connection with an inner lumen and a vacuum supply to provide a vacuum force for grasping tissue in a similar manner to the example described in FIGS. 1A and 1B. Catheter 751 further comprises a rotatable disc 757 having a subset of the distal vacuum apertures 755a. The inner lumen can be designed such that a vacuum can be supplied to the distal vacuum apertures 755a regardless of the orientation of the disc (See FIGS. 7C and 7D). In some implementations, the vacuum lumen is expanded to have the same area as rotatable disc 757 directly underneath the rotatable disc.

To change the axis of the catheter in relationship to the tissue, a vacuum can be applied to the distal vacuum apertures 755a. Further, the vacuum apertures 755b that are outside of rotatable disc 757 can be occluded (FIG. 7E). Any means for occluding vacuum apertures 755b can be utilized. In some implementations, a slidable member within the inner lumen is advanced in the distal direction up to, but not into, the rotatable disc such that all vacuum apertures not within the rotatable disc are occluded. Having vacuum apertures 755b occluded, a vacuum can be supplied to distal vacuum apertures 755a such that only the vacuum apertures within the rotatable disc can grasp the tissue, allowing catheter 751 to be rotated in a circular direction (FIG. 7E).

The various systems for incising tissue can be delivered through the body via a catheter and transcatheter system. The transcatheter system extends proximally to a control system, as is appreciated in the art of transcatheter procedures. A clinician can utilize the control system to advance the transcatheter system through the body to the site where the incision is to be performed. Further, the control system can advance and retract the catheter for performing the incision in the distal and proximal direction in reference to the outer sheath. The control system can be utilized to control the means for incising the tissue. The control system can further comprise a means for regulating the vacuum such that a vacuum can be transferred to the vacuum apertures within the catheter. The control system can further comprise a means for controlling any additional features (e.g., expansion balloon, occluding member) that assist in utilization of the catheter.

While specific configurations of a system for incising tissue is described above with reference to FIGS. 1A to 7C, it should be readily appreciated that various configurations of system for incising tissue can be implemented in any of a variety of combinations of components. Accordingly, the specific configuration of a system for incising tissue described herein should be understood as not to be limited to any specific configuration, but instead can be implemented in any configuration capable of incising tissue.

Various systems for incising tissue can comprise a shape-memory tubular lumen or catheter with a shape-memory lumen, which can be utilized in a transcatheter system. In many instances, the system for incising tissue is utilized for within an inner space within two tissues to incise at least one of the two tissues. At the distal end of the shape-memory tubular catheter, the catheter can bend and curl back in itself using shape-memory, forming loop or a partial loop. The system can further comprise a means for incising tissue, such as a pointed blade, within the lumen of the tubular catheter. The tubular catheter can further comprise a side aperture within a portion of the distal loop. In some implementations, the pointed blade can be utilized to initially pierce a tissue and/or perform an elongated incision of the tissue through the side aperture. Any shape-memory material can be utilized, such as (for example) nitinol.

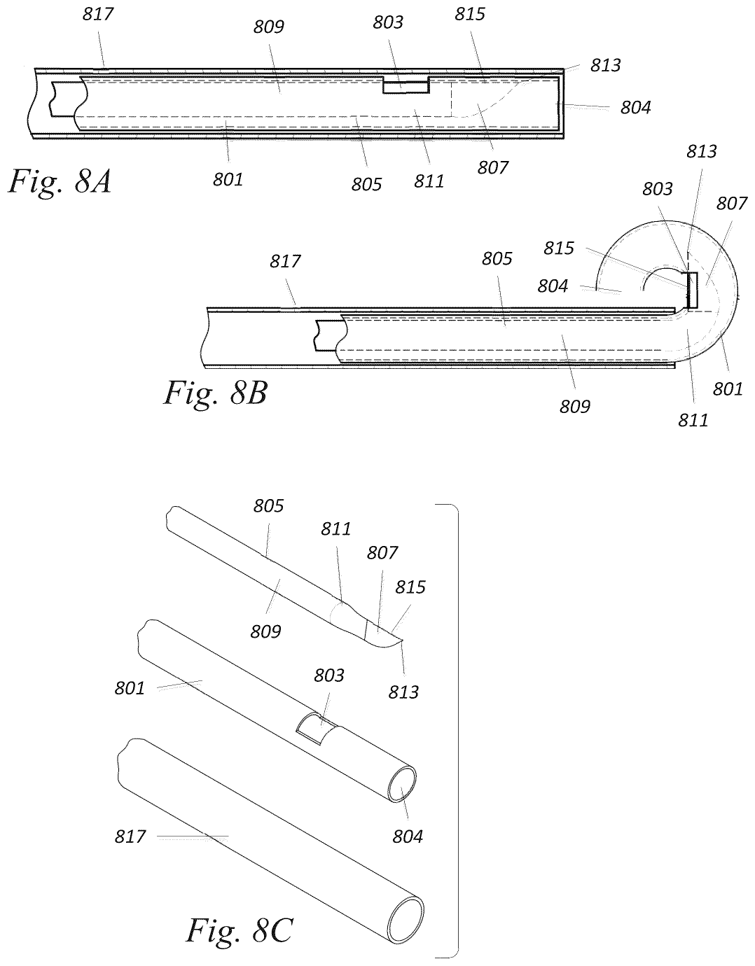

Provided in FIGS. 8A to 8C is an example of system for incising tissue utilizing a shape-memory tubular catheter. The system comprises a shape-memory tubular catheter 801 having an aperture 803 within its side wall. When tubular catheter 801 is in its curled loop shape form (FIG. 8B), aperture 803 faces towards the center of the loop, at a position that is at approximately 90-degrees around the loop from the initial bend of the tubular catheter (i.e., where it begins to curl from the proximal straight portion of the tubular catheter). In some implementations, a vacuum is provided via the inner lumen of tubular catheter 801, which can provide a vacuum via the distal opening 804 or the aperture 803.

Within the lumen of the lumen of tubular catheter 801 is a tool 805 with a blade 807 at the distal end, which can have scalpel-like shape. Tool 805 also includes a shaft 809 proximal to blade 807, which can be elongated and can include a flexible hinge 811 at position adjacent to blade 807, which allows tool 805 to flex and/or bend at the hinge. In some implementations, flexible hinge 811 is able to flex at least at least 90-degrees, allowing a sharp point 813 of blade 807 to be pointed in a direction perpendicular to tool shaft 809 and a sharp edge 815 to be facing back towards the proximal direction of shaft 809. Further, when tubular catheter 801 is in its curled loop shape form, sharp edge 815 aligns with aperture 803 when flexible hinge 811 is flexed 90-degrees such that the sharp edge can be exposed via the aperture.

The exemplary system for incising tissue utilizing a shape-memory tubular catheter can be delivered through the body via a catheter and transcatheter system. The transcatheter system extends proximally to a control system, as is appreciated in the art of transcatheter procedures. A clinician can utilize the control system to advance the transcatheter system through the body to the site where the incision is to be performed. Further, the control system can advance and retract catheter 801 for performing the incision in the distal and proximal direction in reference to an outer sheath 817. Further, outer sheath keeps memory-shaped catheter 801 in a straight conformation as it traverses through the body to the site of incision, where the catheter can be advanced distally to take on its shape-memory. The control system can be utilized to control the tool for incising the tissue. The control system can further comprise a means for regulating the vacuum such that a vacuum can be transferred to the tubular catheter. The control system can further comprise a means for controlling any additional features that assist in utilization of the catheter.

### Methods of Use

The exemplary systems for incising tissue of the disclosure can be utilized in a transcatheter catheter system. In many instances, an incision is made the body and the transcatheter system is inserted therein in order to reach the site of the procedure. In instances in which the pericardial space is to be accessed (e.g., to incise pericardial tissue), a subxiphoid approach can be utilized. Once the catheter reaches the site procedure, the catheter can be advanced distally to perform the incision. In some instances, the method is viewed contemporaneously via a visualization method, such as (for example) sonography or with a camera. In one example the catheter is configured to be purged with saline to assist in the visualization and/or to minimize heating of the tissue being incised and/or provide lubrication.

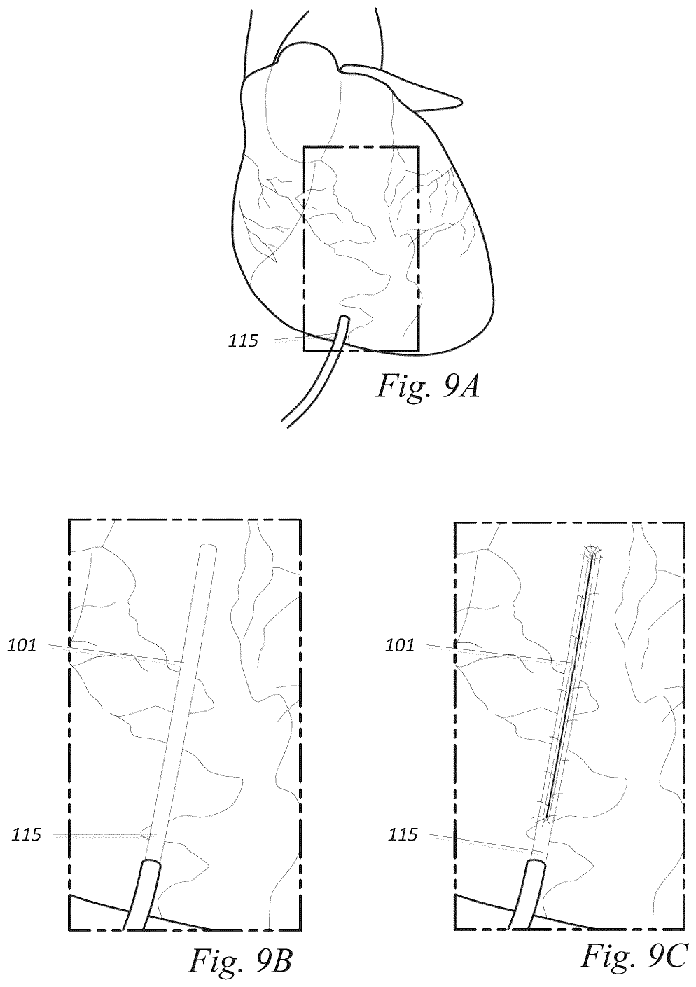

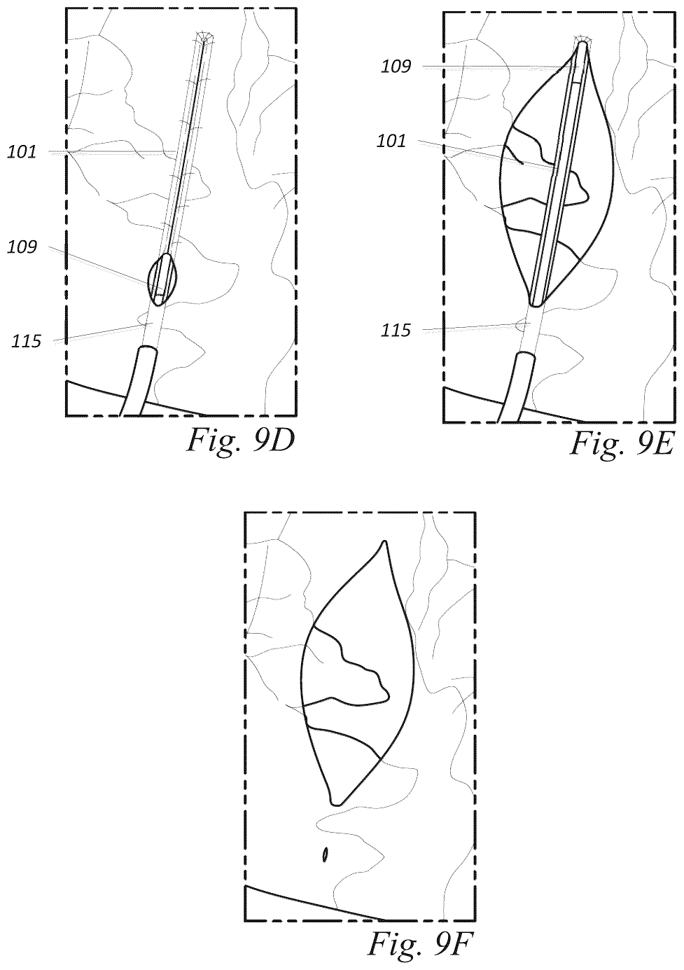

Provided in FIGS. 9A to 9F is an exemplary method of utilizing a system for incising a tissue, utilizing the pericardium as an example. The system utilized in this exemplary method is depicted in FIGS. 1A and 1B, but any of the exemplary systems as described for FIGS. 1A to 7C can utilize a similar methodology with various alterations as dependent on the various features of the various systems, which can be readily appreciated by those skilled in the art. Catheter systems can be selected for particular surgical outcomes. For instance, the selection of an incising means may be based on the tissue type to be incised. In some instances, a blade may be preferred and selected. In some instances, a cauterized wire may be preferred and selected. Further, a catheter system may be selected based on its size, as the indentation with vacuum apertures can predetermine the amount of tissue to be incised and removed.

FIG. 9A depicts a heart that is covered in pericardium. A transcatheter system delivers the system for incising tissue into the pericardial space by creating an incision in the pericardium and traversing through the pericardium (FIG. 9A). FIGS. 9B to 9F focus in on the rectangular box depicted in FIG. 9A.

FIG. 9B depicts the outer sheath 115 of the transcatheter system penetrating the pericardium, with the distal portion of outer sheath 115 within the pericardial space. Catheter 101 is advanced distally out of outer sheath 101 with the indentation and vacuum apertures facing the pericardium. At this stage, an expandable balloon can be utilized to increase the catheter profile and bring the indentation and vacuum apertures in closer proximity to the pericardium. A vacuum is applied, pulling the pericardium into the indentation (FIG. 9C). If a different angle of incision is desired, a rotatable disc comprising a subset of vacuum apertures at the distal end of the aperture can be utilized to grasp the pericardium and the catheter can be rotated to the desired angle.

To begin the incision, the leading-edge point of blade 109 pierces the pericardium by advancing the blade in a distal direction (FIG. 9D). The blade is continued to be advanced in the distal direction to incise the tissue (FIG. 9E). Lancets, prongs, corkscrews, or other components that can enhance the grasp of tissue can optionally be utilized as a counter force can be utilized as a counter force. In some implementations, the incision is performed utilizing a wire, which can optionally conduct electricity and/or heat. In some implementations, one or more rotational blades are utilized to incise the tissue.

After the incision is complete, the incised tissue remains within the indented portion of catheter 101 with the vacuum supply still active. The catheter and tissue are proximally retracted out of the pericardial space and removed from the patient, leaving an incision within the pericardial tissue (FIG. 9F).

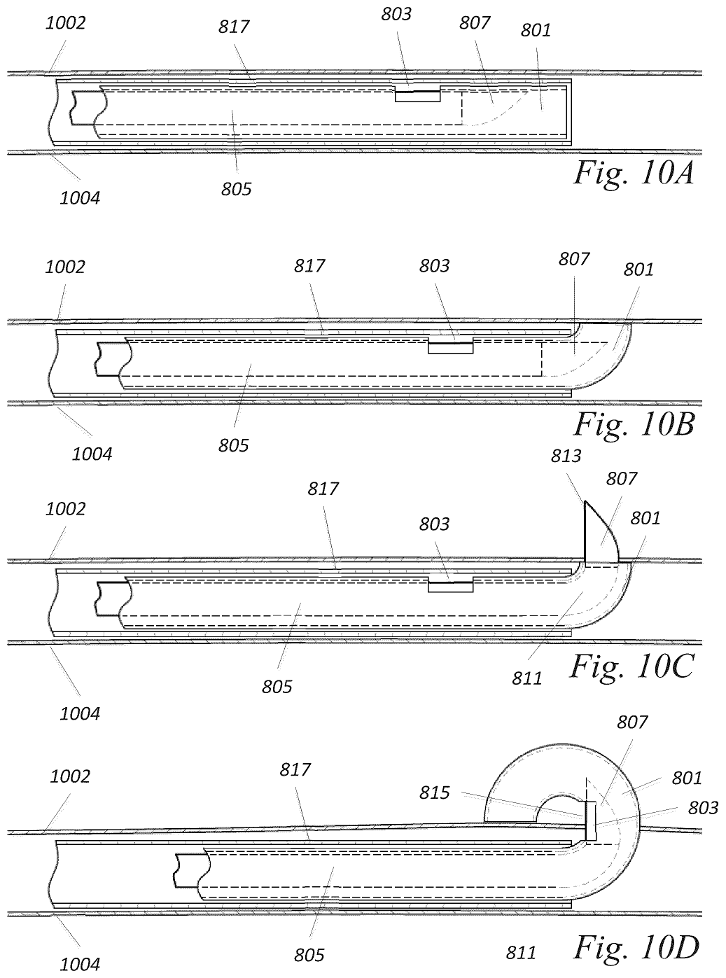

Provided in FIGS. 10A to 10D is an exemplary method utilizing a system for incising tissue comprising shape-memory tubular catheter and blade. The exemplary system utilized in this exemplary method is depicted in FIGS. 8A and 8B. This exemplary method shows the catheter system between two tissue layers, which can be any two layers of tissue, such as (for example) the pericardium and myocardium of the heart.

Catheter 801 is positioned between a first layer of tissue 1002 and a second layer of tissue 1004. When delivered to the site of incision, catheter 801 is confined within outer sheath 817 and blade 807 is within catheter 801 (FIG. 10A). Catheter 801 is distally advanced such that the distal end of the catheter is exposed. Catheter 801 comprises shape-memory material and the shape-memory material is configured with a curl. Shape-memory material of catheter 801 is constrained within a retractable outer sheath 817 for delivery to the incision site. At the incision site, outer sheath 817 is retracted and the distal end of catheter 801 begins to curl. Catheter 801 is positioned within outer sheath 817 such that that the distal end of the catheter bends or curls towards the tissue to be incised (FIG. 10B). In some situations, a vacuum force can be transmitted through catheter 801 via the distal opening, grasping tissue 1002. Notably, the bulk of the vacuum force is applied via the distal end of catheter 801 due to the fact that aperture 803 in the side wall of the catheter is covered by outer sheath 817.

To begin incision in tissue 1002, tool 805 is distally advanced to and through the distal end of catheter 801. Flexible hinge 811 allows tool 805 to curl in complementary fashion to that of the curl of the distal end of catheter 801, which results in sharp point 813 of blade 807 facing towards tissue 1002. Flexible hinge 811, or alternatively, tool 805 is composed of a shape-memory material component to allow the blade 807 to curl in a complimentary fashion with the lumen of catheter 801. Sharp point 813 can penetrate and traverse through tissue 1002 as it distally advances though the distal end of catheter 801 (FIG. 10C). In some instances, the vacuum force applied via the distal end of catheter 801 provides a counterforce to assist in sharp point 813 penetration and traversal through tissue 1002.

Once tissue 1002 is traversed by blade 807, catheter 801 is further distally advanced such that the distal end of the shape-memory lumen of catheter traverses through the tissue. As the distal end of catheter 801 is advanced beyond outer sheath 817 and through tissue 1002, it curls utilizing its shape-memory such that the aperture at the distal end of the catheter is presented to and straddles tissue 1002 on opposing sides of tissue 1002 (FIG. 10D). For example, catheter 801 traverses approximately thru a 180 degree arc while aperture travels approximately thru a 90 degree arc relative to the proximal-distal longitudinal axis of the sheath. Outer sheath 817 and the proximal portion of catheter 801 remain on the initial side of tissue 1002. Thus, aperture 801 in the side wall of catheter 801 is distally advanced out of outer sheath 817 and aligns with tissue 1002 such that the face of the aperture is substantially perpendicular to the plane of the tissue 1002. Aperture 803 is radially configured such that tissue 1002 can be presented to sharp edge 815 of blade 807. Sharp edge 815 of blade 807 is positioned to face towards aperture 803. In some situations, a vacuum force is applied, resulting in both the vacuum force to be distributed at the distal end of the catheter and at aperture 803. The vacuum force can help stabilize the tissue 1002 and/or draw and present tissue 1002 to sharp edge 815 and/or provide tensioning of tissue 1002 and/or a counter force for incising the tissue 1002.

With blade 807 in position to incise the tissue, the catheter system inclusive of the outer sheath 817, catheter 801, and tool 805, is proximally retracted in concert such that the components remain in relative position with one another. The proximal retraction of the catheter system results in blade 807 being pulled through and incising tissue 1002. Once the incision is complete, tool 805 is retracted into catheter 801 and the catheter is distally retracted into outer sheath 817. The catheter system can be relocated to another position to perform another incision or proximally retracted out of the subject. In one example, a cauterizing or cutting wire can be substituted for, or used in combination with, sharp edge 815. The system depicted in FIGS. 10A to 10D can further be configured with purging means for cooling tissue and/or displacing tissue during incising.

### DOCTRINE OF EQUIVALENTS

While the above description contains many specific embodiments, these should not be construed as limitations on the scope of the disclosure, but rather as an example of one embodiment thereof. Accordingly, the scope of the disclosure should be determined not by the embodiments illustrated, but by the appended claims and their equivalents.

What is claimed is:

1. A system for incising tissue comprising a catheter having a proximal-distal axis extending from a distal end of the catheter, said catheter comprising:
an indentation adjacent to the distal end of the catheter and comprising a length dimension extending along the proximal-distal axis of the catheter and a width dimension transverse to the proximal-distal axis of the catheter;
a vacuum system comprising:
one or more vacuum apertures situated within the indentation; and
a vacuum lumen extending along the proximal-distal axis of the catheter and comprising a distal end connected to the one or more vacuum apertures and a proximal end configured for connection to a vacuum source, wherein said vacuum system, when activated, is configured to pull tissue into the indentation; and
means for incising tissue connected adjacent to the distal end of the catheter and slidable relative to the indentation, whereby tissue located in the indentation can be incised by said means for incising tissue by sliding said means for incising relative to the indentation, while the vacuum system is activated.

2. The system of claim 1, wherein the catheter further comprises means for providing a counterforce located in the indentation and configured to engage tissue located in the indentation and provide a force that counters a force exerted by said means for providing an incision.

3. The system of claim 2, wherein means for providing a counter force is one or more of: a lancet, a prong, a helix, a barb, a clasp, a jagged edge, or a textured surface.

4. The system of claim 1, wherein the vacuum system further comprises a slidable member slidably connected to the catheter and configured to selectively occlude one or more of the vacuum apertures to thereby control an amount of vacuum applied to the tissue.

5. The system of claim 1, wherein the catheter further comprises a rotatable disc located in the indentation, wherein one or more of the vacuum apertures are situated within the rotatable disc.

6. The system of claim 1, wherein the catheter further comprises:
a rotatable disc located in a first section of the indentation comprising one or more of the vacuum apertures, wherein the rotatable disc is rotatable relative to the catheter;
a second section of the indentation adjacent the first section comprising one or more of the vacuum apertures; and
a slidable member slidably connected to the catheter and configured to selectively occlude the one or more vacuum apertures located in the second section of the indentation,
such that when the vacuum system is activated and the slidable member is positioned to occlude the one or more vacuum apertures located in the second section of the indentation, the catheter may be rotated relative to tissue adjacent the catheter via the rotatable disc and engagement of the one or more vacuum apertures located in the rotatable disc with the tissue.

7. The system of claim 1, wherein at least one of the length or width dimension of the indentation determines an amount of tissue to be incised.

8. The system of claim 1, wherein means for incising tissue is a blade in connection with the catheter, the blade having a sharpened edge and capable of moving in a direction along the proximal-distal axis relative to the indentation.

9. The system of claim 8, wherein the blade comprises a leading-edge point on the sharpened edge, wherein the sharpened edge curves or angles back from the leading-edge point.

10. The system of claim 1, wherein means for incising tissue is a wire in connection with the catheter, the wire is configured to move in a direction along the proximal-distal axis relative to the indentation and is heat conductive or electrically conductive.

11. The system of claim 1, wherein means for incising tissue is a first blade in connection with the catheter, the first blade having the sharpened edge and capable of rotating in a direction perpendicular the proximal-distal axis of the catheter relative to the indentation.

12. The system of claim 11, wherein the catheter further comprises a rotating member comprising a blunt edge in connection with the catheter for compressing tissue.

13. The system of claim 11 further comprising a second blade in connection with the catheter, the second blade having the sharpened edge and capable of rotating in a direction perpendicular the proximal-distal axis and opposite to the first blade.

14. The system of claim 1, wherein the catheter further comprises one or expandable means located adjacent to the distal end of the catheter for increasing a diameter of the catheter.

15. The system of claim 1, wherein the catheter further comprises one or expandable means located adjacent to the distal end of the catheter and each respectively spaced apart a distance from the indentation for providing support for the distal end of the catheter relative to tissue surrounding the catheter.

16. The system of claim 1 further comprising a control system, wherein the catheter and the control system are a part of a transcatheter system such that the control system is in operable connection with the catheter and is capable of distally advancing and proximally retracting the catheter along the proximal-distal axis; wherein the control system is further capable of operating means for incising tissue.

17. A system for incising tissue comprising a catheter having a proximal-distal axis extending between from a distal end of the catheter, said catheter comprising:
an indentation adjacent to the distal end of the catheter and comprising a length dimension extending along the proximal-distal axis of the catheter and a width dimension transverse to the proximal-distal axis of the catheter;
a vacuum system comprising:
one or more vacuum apertures situated within the indentation; and
a vacuum lumen extending along the proximal-distal axis of the catheter and comprising a distal end connected to the one or more vacuum apertures and a proximal end configured for connection to a vacuum source, wherein said vacuum system, when activated, is configured to pull tissue into the indentation;
means for incising tissue connected adjacent to the distal end of the catheter and slidable relative to the indentation, whereby tissue located in the indentation can be incised by said means for incising tissue by sliding said means for incising tissue relative to the indentation, while the vacuum system is activated;
means for providing a counterforce located in the indentation and configured to engage tissue located in the indentation and provide a force that counters a force exerted by said means for providing an incision; and
a slidable member slidably connected to the catheter and configured to selectively occlude one or more of the vacuum apertures to thereby control an amount of vacuum applied to the tissue.

18. The system of claim 17, wherein at least one of the length or width dimension of the indentation predetermines an amount of tissue to be incised.

19. A system for incising tissue comprising a catheter having a proximal-distal axis extending between from a distal end of the catheter, said catheter comprising:
an indentation adjacent to the distal end of the catheter and comprising a length dimension extending along the proximal-distal axis of the catheter and a width dimension transverse to the proximal-distal axis of the catheter;
a vacuum system comprising:
one or more vacuum apertures situated within the indentation; and
a vacuum lumen extending along the proximal-distal axis of the catheter and comprising a distal end connected to the one or more vacuum apertures and a proximal end configured for connection to a vacuum source, wherein said vacuum system, when activated, is configured to pull tissue into the indentation;
means for incising tissue connected adjacent to the distal end of the catheter and slidable relative to the indentation, whereby tissue located in the indentation can be incised by said means for incising tissue by sliding said means for incising relative to the indentation, while the vacuum system is activated; and
a rotatable disc located in a first section of the indentation, wherein the rotatable disc is rotatable relative to the catheter.

20. The system of claim 19, wherein the catheter further comprises:
one or more vacuum apertures located in the rotatable disc;
a second section of the indentation adjacent the first section comprising one or more of the vacuum apertures; and
a slidable member slidably connected to the catheter and configured to selectively occlude the one or more vacuum apertures located in the second section of the indentation,
such that when the vacuum system is activated and the slidable member is positioned to occlude the one or more vacuum apertures located in the second section of the indentation, the catheter may be rotated relative to tissue adjacent the catheter via the rotatable disc and engagement of the one or more vacuum apertures located in the rotatable disc with the tissue.
